(19) United States
(12) Patent Application Publication
(10) Pub. No.: US 2026/0335106 A1
(43) Pub. Date: Oct. 29, 2026

(54) SYSTEMS AND METHODS FOR CONTROLLING A LOWER LIMB PROSTHETIC DEVICE

(71) Applicant: Össur Iceland ehf, Reykjavík (IS)

(72) Inventors: Atli Orn Sverrisson, Reykjavik (IS); Hao Geng, Reykjavik (IS); Stefan Pall Sigurthorsson, Reykjavik (IS); Arni Einarsson, Reykjavik (IS)

(21) Appl. No.: 19/491,171

(22) PCT Filed: Dec. 12, 2024
(86) PCT No.: PCT/IB2024/055726

§ 371 Date: Dec. 8, 2025

Related U.S. Application Data
(60) Provisional application No. 63/507,927, filed on Jun. 13, 2023.
(60) Provisional application No. 63/583,046, filed on Sep. 15, 2023.

(51) Int. Cl. A61F 2/70; A61B 5/00; A61B 5/11

(52) U.S. Cl. CPC A61F 2/70; A61B 5/112; A61B 5/4851; A61B 5/6811; A61B 5/7203; A61B 5/725; A61B 2562/0219; A61F 2002/704

(57) ABSTRACT

Systems and methods are provided for estimating the attitude, shank angle, velocity, and/or position of a lower limb prosthetic device based on data or measurements from an IMU. The systems and methods may reduce or minimize drift. The estimated attitude, shank angle, velocity, and/or position of the lower limb prosthetic may be used to control one or more functions of the lower limb prosthetic device.

### INCORPORATION BY REFERENCE TO ANY PRIORITY APPLICATIONS

The present application is a National Phase Application of PCT/IB2024/055726 filed Jun. 12, 2024, titled SYSTEMS AND METHODS FOR CONTROLLING A LOWER LIMB PROSTHETIC DEVICE, which claims priority to U.S. Provisional Application No. 63/583,046, filed Sep. 15, 2023, and U.S. Provisional Application No. 63/507,927, filed Jun. 13, 2023, which are incorporated herein by reference. Any and all applications for which a foreign or domestic priority claim is identified in the Application Data Sheet as filed with the present application are hereby incorporated by reference under 37 CFR 1.57.

### BACKGROUND

### Field

The present application relates to estimating the orientation of a lower limb prosthetic, and more particularly, systems and methods for reducing or minimizing drift in estimations generated with data from inertial measurement units (IMUs).

### Description of the Related Art

Lower limb prosthetic devices are designed to restore the mobility and functionality of amputees who have lost their lower limbs due to injury, disease, or congenital defects. Active prosthetic devices use motors and sensors to adjust the device operation according to the user's intention and environment. Inertial measurement units (IMUs) are commonly used sensors in prosthetic devices, as they can measure the motion of the device using gyroscopes and accelerometers. However, IMU-based methods suffer from drifts and noise due to measurement errors, which can affect the accuracy and reliability of the motion estimation.

### SUMMARY

For purposes of this summary, certain aspects, advantages, and novel features are described herein. It is to be understood that not necessarily all such advantages may be achieved in accordance with any particular aspect. Thus, for example, those skilled in the art will recognize the disclosures herein may be carried out in a manner that achieves one or more advantages taught herein without necessarily achieving other advantages as may be taught or suggested herein.

In some aspects, a control system may improve the functionality and performance of prosthetic devices.

In some aspects, the control system may estimate an attitude, velocity, and/or a position of a lower limb prosthetic device based on data or information from an IMU. In some aspects, the systems and methods may use a Kalman filter to fuse data from a gyroscope and an accelerometer. In some aspects, the control system may estimate an attitude, velocity and/or a position of the lower limb prosthetic device base on a gait state of the lower limb prosthetic device to reduce drift or errors in the estimated attitude, velocity and/or position of the lower limb prosthetic device.

In some aspects, a method for tracking a shank angle of a lower limb prosthetic device may include receiving an angular velocity vector of a shank of the lower limb prosthetic device from a gyroscope, an acceleration vector of the shank from an accelerometer, and a force applied to the lower limb prosthetic device from a force sensor; determining a gait state of the lower limb prosthetic device based at least in part on the angular velocity vector, the acceleration vector, and the force; generating an external acceleration vector of the shank based on the gait state of the lower limb prosthetic device; and generating a shank angle vector of the shank using a Kalman filter to fuse the angular velocity vector, the acceleration vector, and the external acceleration vector.

In some aspects, a method for tracking a shank angle of a lower limb prosthetic device may include generating an acceleration sum vector of a stride, wherein determining the acceleration sum vector includes: receiving a gravity vector of a shank of a lower limb prosthetic device and an acceleration vector of the shank of the lower limb prosthetic device; determining a shank angle of the shank based at least in part on the gravity vector; rotating the acceleration vector to a stride reference frame using the shank angle to generate a rotated acceleration vector; adding the rotated acceleration vector to the acceleration sum vector; generating an average acceleration vector of the stride based on the acceleration sum vector; generating a vector angle of the average acceleration vector; and generating a compensated shank angle by subtracting the vector angle from a shank angle of the shank at an end of the stride.

In some aspects, a method for tracking a shank angle of a lower limb prosthetic device may include receiving sensor data from an accelerometer, a gyroscope, and force sensors; determining a gait state of the lower limb prosthetic device based at least in part on the sensor data; generating an external acceleration vector of a shank of the lower limb prosthetic based on the gait state of the lower limb prosthetic device; and generating a shank angle vector of the shank based at least in part on the sensor data from the accelerometer and the gyroscope, and the external acceleration vector.

In some aspects, the sensor data may include an acceleration vector of the shank, an angular velocity vector of the shank, and a force applied to the lower limb prosthetic device.

In some aspects, a Kalman filter may be used to generate the shank angle vector.

In some aspects, the method may include controlling one or more functions of the lower limb prosthetic device based on the shank angle vector.

In some aspects, the gait state may include a stationary state, a single supported state or an others state.

In some aspects, if the gait state is the stationary state, the external acceleration vector may be zero, if the gait state is the single supported state, the external acceleration vector may be generated using an inverted pendulum model, and wherein if the gait state is the others state, the external acceleration vector is generated using a low pass filtered model.

In some aspects, a method for tracking a shank angle of a lower limb prosthetic device may include receiving sensor data from an accelerometer and a gyroscope; determining a shank angle of the shank based at least in part on the sensor data from the gyroscope; generating a vector angle based on average acceleration values of a stride; and generating a compensated shank angle by subtracting the vector angle from the shank angle.

In some aspects, the method may include generating an acceleration sum vector of the stride.

In some aspects, the method may include generating average acceleration values of the stride based on the acceleration sum vector.

In some aspects, average acceleration values of the stride may be a quotient of the acceleration sum vector of the stride and a number of times sensor data is received during the stride.

In some aspects, the method may include controlling one or more functions of the lower limb prosthetic device based on the shank angle vector.

In some aspects, a method for tracking a lower limb prosthetic device may include receiving sensor data from an accelerometer, a gyroscope, and force sensors; determining a shank angle of a shank of the lower limb prosthetic; generating a converted external acceleration vector based on an external acceleration of the shank; and generating a velocity of the lower limb prosthetic device based on the converted external acceleration vector and a correction factor.

In some aspects, the method may include generating a position of the lower limb prosthetic device based on the velocity of the lower limb prosthetic.

In some aspects, the method may include controlling one or more functions of the lower limb prosthetic device based on the position of the lower limb prosthetic.

In some aspects, the method may include generating the correction factor based on a gait state of the lower limb prosthetic device.

In some aspects, the gait state may include a stationary state, a single supported state or an others state.

In some aspects, if the gait state is the stationary state or the single supported state, the correction factor may be determined based on a correction factor of a previous gait cycle, wherein if the gait state is the others state, the correction factor may be a correction factor determined in a previous single supported state.

In some aspects, the method may include updating the correction factor when the lower limb prosthetic is in a single supported stance phase.

In some aspects, the method may include controlling one or more functions of the lower limb prosthetic device based on the velocity of the lower limb prosthetic.

In some aspects, a method for tracking a shank angle of a lower limb prosthetic device may include receiving sensor data from an accelerometer, a gyroscope, and a knee angle sensor; determining a gait state of the lower limb prosthetic device based at least in part on the sensor data; generating an external acceleration vector of a shank of the lower limb prosthetic based on the gait state of the lower limb prosthetic device; and generating a shank angle vector of the shank based at least in part on the sensor data from the accelerometer and the gyroscope, and the external acceleration vector.

In some aspects, the sensor data may include an acceleration vector of the shank, an angular velocity vector of the shank, and a joint angle of a knee joint of the lower limb prosthetic device.

In some aspects, a Kalman filter may be used to generate the shank angle vector.

In some aspects, the method may include controlling one or more functions of the lower limb prosthetic device based on the shank angle vector.

In some aspects, the gait state may include a stationary state, a single supported state or an others state.

In some aspects, if the gait state is the stationary state, the external acceleration vector may be zero, if the gait state is the single supported state, the external acceleration vector may be generated using an inverted pendulum model, and wherein if the gait state is the others state, the external acceleration vector is generated using a low pass filtered model.

In some aspects, the gait state may be the single supported state when the acceleration vector is equal to a gravity vector and the joint angle of the knee joint indicates that the knee joint is fully extended.

In some aspects, a method for tracking a shank angle of a lower limb prosthetic device may include receiving sensor data from an accelerometer, a gyroscope, and at least a third sensor, wherein the third sensor comprises a plurality of force sensors, or a knee angle sensor; determining a gait state of the lower limb prosthetic device based at least in part on the sensor data; generating an external acceleration vector of a shank of the lower limb prosthetic based on the gait state of the lower limb prosthetic device; and generating a shank angle vector of the shank based at least in part on the sensor data from the accelerometer and the gyroscope, and the external acceleration vector.

In some aspects, the sensor data may include an acceleration vector and an angular velocity vector of the shank.

In some aspects, the third sensor may include the plurality of force sensors and the sensor data may further include a force applied to the lower limb prosthetic device.

In some aspects, the third sensor may include the knee angle sensor and the sensor data may further include a knee angle.

In some aspects, a Kalman filter may be used to generate the shank angle vector.

In some aspects, the method may include controlling one or more functions of the lower limb prosthetic device based on the shank angle vector.

In some aspects, the gait state may include a stationary state, a single supported state, or an others state.

In some aspects, if the gait state is the stationary state, the external acceleration vector may be zero, if the gait state is the single supported state, the external acceleration vector may be generated using an inverted pendulum model, and wherein if the gait state is the others state, the external acceleration vector is generated using a low pass filtered model.

In some aspects, the third sensor may include the knee angle sensor, and the single supported state may be assumed based on a knee angle indicative of a fully extended knee and accelerometer data being equal to gravity for a predetermined duration.

In some aspects, a method for tracking a lower limb prosthetic device may include receiving sensor data from an accelerometer, a gyroscope, and at least a third sensor, wherein the third sensor comprises a plurality of force sensors or a knee angle sensor; determining a shank angle of a shank of the lower limb prosthetic; generating a converted external acceleration vector based on an external acceleration of the shank; and generating a velocity of the lower limb prosthetic device based on the converted external acceleration vector and a correction factor.

In some aspects, the method may include generating a position of the lower limb prosthetic device based on the velocity of the lower limb prosthetic.

In some aspects, the method may include controlling one or more functions of the lower limb prosthetic device based on the position of the lower limb prosthetic.

In some aspects, the method may include generating the correction factor based on a gait state of the lower limb prosthetic device.

In some aspects, the gait state may include a stationary state, a single supported state or an others state.

In some aspects, if the gait state is the stationary state or the single supported state, the correction factor may be determined based on a correction factor of a previous gait cycle, wherein if the gait state is the others state, the correction factor may be a correction factor determined in a previous single supported state.

In some aspects, the method may include updating the correction factor when the lower limb prosthetic is in a single supported stance phase.

In some aspects, the method may include controlling one or more functions of the lower limb prosthetic device based on the velocity of the lower limb prosthetic.

In some aspects, the third sensor may include the knee angle sensor, and the single supported state may be assumed based on a knee angle indicative of a fully extended knee and accelerometer data being equal to gravity for a predetermined duration.

In some aspects, the third sensor may include the plurality of force sensors and the sensor data further comprises a force applied to the lower limb prosthetic device.

In some aspects, the third sensor may include the knee angle sensor and the sensor data may further include a knee angle.

### BRIEF DESCRIPTION OF THE DRAWINGS

These and other features, aspects, and advantages of the present disclosure are described with reference to the drawings of certain embodiments, which are intended to schematically illustrate certain embodiments and not to limit the disclosure.

FIG. 1A illustrates schematically a control system of a lower limb prosthetic device.

FIG. 1B illustrates a prosthetic knee component with the control system.

FIG. 1C illustrates example components of an above knee lower limb prosthetic device.

FIG. 2A illustrates a schematic of a gait cycle.

FIG. 2B illustrates a schematic of forces measured by force sensors of the control system of FIG. 1A throughout a gait cycle.

FIG. 2C illustrates a schematic of a lower limb prosthetic device in a single support state.

FIG. 3A is a flowchart illustrating an example process for determining a shank angle of a lower limb prosthetic device.

FIGS. 3B and 3C are example block diagrams illustrating control systems for determining a shank angle of a lower limb prosthetic device.

FIG. 4A is a flowchart illustrating an example process for determining a velocity and/or position of the lower limb prosthetic device

FIG. 4B is an example block diagram illustrating a control system for determining a velocity and a position of a lower limb prosthetic device.

FIG. 5A is a block diagram illustrating a control system for determining reducing or minimizing drift in a shank angle estimation of a lower limb prosthetic device.

FIG. 5B is a flowchart illustrating a process for generating a compensated shank angle of a lower limb prosthetic device.

FIG. 6 is a flowchart illustrating a process for controlling one or more functions of a lower limb prosthetic device.

FIGS. 7A and 7B illustrate schematically control systems of a lower limb prosthetic device.

FIG. 8 illustrates another prosthetic knee component with a knee angle sensor.

### DETAILED DESCRIPTION

Although several aspects, examples, and illustrations are disclosed below, it will be understood by those of ordinary skill in the art that the system, methods, and devices described herein extend beyond the specifically disclosed aspects, examples, and illustrations and includes other uses of the system, methods, and devices and obvious modifications and equivalents thereof. The terminology used in the description presented herein is not intended to be interpreted in any limited or restrictive manner simply because it is being used in conjunction with a detailed description of certain specific aspects of the disclosure. In addition, aspects of the disclosure can comprise several novel features and no single feature is solely responsible for its desirable attributes or is essential to practicing the system, methods, and devices herein described.

Active prosthetic devices can improve the quality of life of amputees. Active prostheses include a control system that can control the prosthesis to approximate motion of a missing limb. In order to control the device operation, lower limb prosthetics can use inertial measurement units (IMU) or some form of inertial sensing, such as gyroscopes and accelerometers, to collect information about the gait and the environment in which a user is moving. The IM can include at least an accelerometer and a gyroscope for measuring angular rates and accelerations, which can be used for attitude and position estimation. However, estimations or calculations made based on information or data from IMUs can suffer from drift caused by the accumulated stochastic errors due to noise in signals generated by the IMU. In accordance with several aspects, the systems and methods described herein advantageously reduce or minimize the drift in estimations or calculation made based on information or data from the IMU on the lower limb prosthetic device.

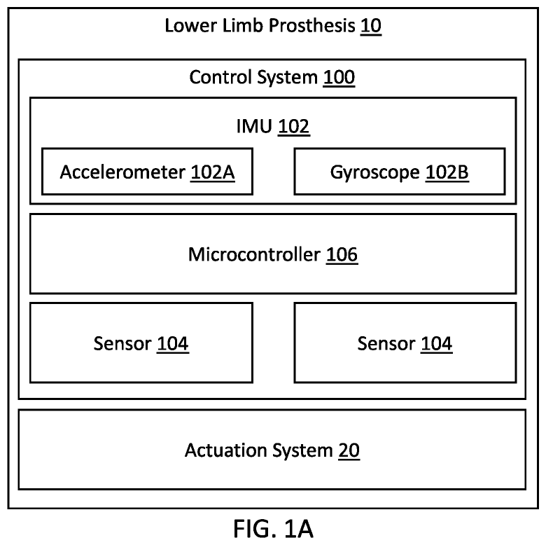

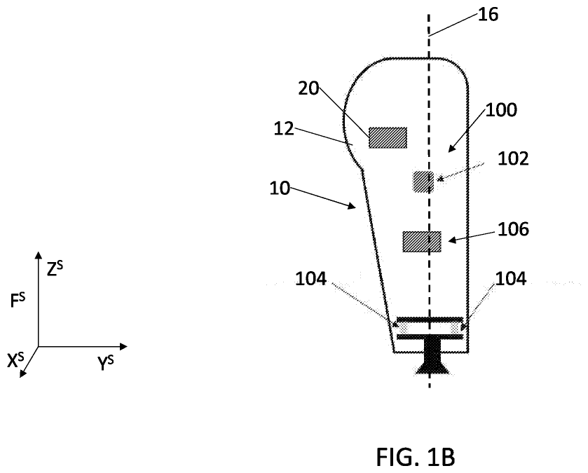

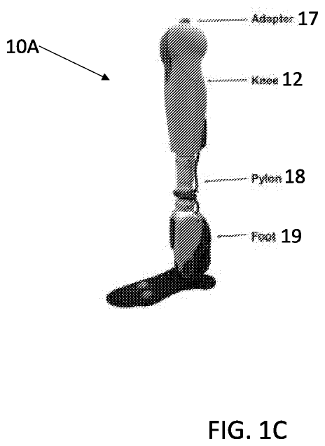

FIGS. 1A and 1B illustrate an example lower limb prosthetic device or prosthesis 10 with a control system 100. The lower limb prosthetic device 10 may include one or more actuation systems 20. The one or more actuation systems 20 may include one or more actuators and/or one or more dampeners. The one or more actuation system 20 may control or modify rotation the lower limb prosthetic device 10 throughout a gait cycle. In some aspects, such as shown in FIG. 1B, the control system 100 may be positioned in a knee component 12 of the lower limb prosthetic device 10. Alternatively, the control system may be positioned in a different component of the lower limb prosthetic device 10. For example, as illustrated in FIG. 1C, a lower limb prosthesis 10A for an above knee amputees, also known as an above knee or transfemoral prosthesis, can include a knee component 12, a pylon component 18 (i.e., a shin portion or shaft), and/or a prosthetic foot component 19. An adapter 17 at a proximal end of the knee component 12 can be coupled to a residual limb of the amputee.

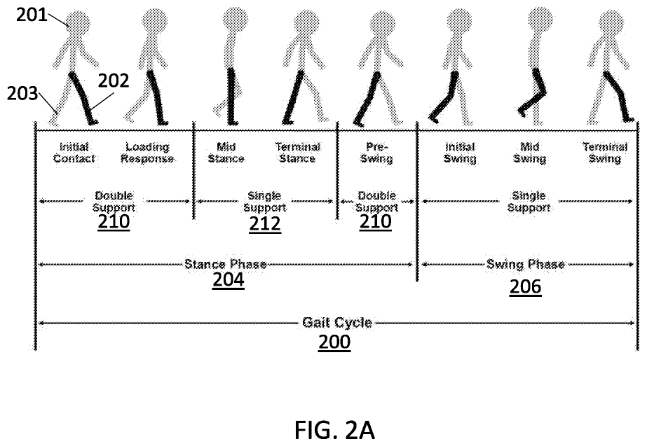

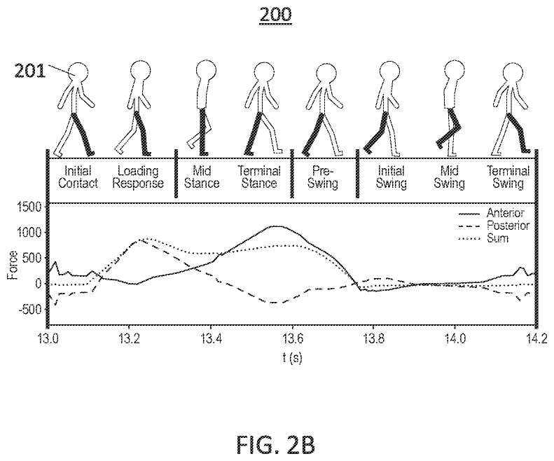

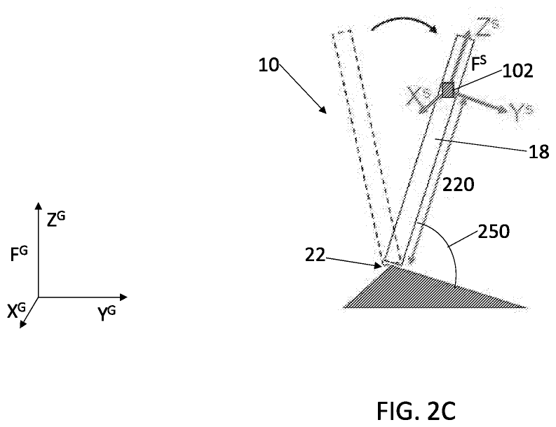

The control system 100 may include an inertial measurement unit (IMU) 102. In some aspects, the IMU 102 may include at least an accelerometer 102A and/or a gyroscope 102B. The accelerometer 102A may measure or determine an acceleration (i.e., linear acceleration) with reference to a body-fixed frame F^{S} (i.e., sensor frame) (shown in FIG. 2C). The body-fixed frame F^{S} may include a shaft x-axis X^{S} (i.e., an axis in a direction into and out of the page), the shaft y-axis Y^{S} (i.e., an axis perpendicular to the length of the lower limb prosthetic device 10) and the shaft z-axis Z^{S} (i.e., an axis along the length of the lower limb prosthetic device 10). The IMU 102 may include one (1) accelerometer 102A, two (2) accelerometers 102A, and/or three (3) accelerometers 102A. In some aspects, the accelerometer 102A may determine or measure the acceleration in one direction. In some aspects, each accelerometer 102A may determine or measure the acceleration in a different direction. For example, a first accelerometer 102A may determine or measure the acceleration in a direction of the shaft x-axis X^{S}, a second accelerometer 102A may determine or measure the acceleration in a direction of the shaft y-axis Y^{S}, and a third accelerometer 102A may determine or measure the acceleration in a direction of the shaft z-axis Z^{S}. In some aspects, the accelerometer 102A may include a 3-axis accelerometer, and the accelerometer 102A may determine or measure the acceleration in the direction of the shaft x-axis X^{S}, the direction of the shaft y-axis Y^{S}, and the direction of the shaft z-axis Z^{S}. However, during dynamic conditions, the accelerometer 120A measures not only the gravity, but also the external acceleration, thus making it difficult to distinguish them from each other. Since the accelerometer readings are relative to the sensor's reference frame, the distinction between the two becomes difficult as the gravity component changes with sensor orientation. Without further correction by separating the external acceleration from measurements of effect due to the gravity, IMU-based navigation methods, also referred to as dead reckoning, can accumulate errors and rapidly cause significant drifts. This can be due to the IMU-based methods using integration to calculate velocity and position. The disclosure herein provides systems and methods for reducing such drifts.

The gyroscope 102B may measure or determine an angular velocity with reference to the body-fixed frame F^{S}. The IMU 102 may include one (1) gyroscope 102B, two (2) gyroscopes 102B, and/or three (3) gyroscopes 102B. In some aspects, the gyroscope 102B may determine or measure the angular velocity in one direction. In some aspects, each gyroscope 102B may determine or measure the angular velocity in a different direction. For example, a first gyroscope 102B may determine or measure the angular velocity in the direction of the shaft x-axis X^{S}, a second gyroscope 102B may determine or measure the angular velocity in the direction of the shaft y-axis Y^{S}, and a third gyroscope 102B may determine or measure the angular velocity in the direction of the shaft z-axis Z^{S}. In some aspects, the gyroscope 102B may include a 3-axis gyroscope, and the gyroscope 102B may determine or measure the angular velocity in the direction of the x-axis X^{S}, the direction of the shaft y-axis Y^{S}, and the direction of the shaft z-axis Z^{S}. Similar to the challenges described herein with reference to the accelerator measurements, when integrating the gyroscope signal, the measurement errors are also integrated, leading to unbounded orientation drift errors. The disclosure herein provides systems and methods for reducing such drifts.

In some aspects, the control system 100 may include sensors 104 in addition to the IMU 102. The control system 100 may include one (1) sensor 104, two (2) sensors 104, three (3) sensors 104, four (4) sensors 104, five (5) sensors 104, and/or six (6) sensors. The sensors 104 may include a force sensor for measuring or determining a force F applied to the lower limb prosthetic device 10. In some aspects, the force F applied to the lower limb prosthetic device 10 may include a force applied along a longitudinal axis 16 of the lower limb prosthetic device 10. For example, the force F may include a force applied to the lower limb prosthetic device 10 when the foot portion of the lower limb prosthetic device 10 contacts a walking surface. The sensors 104 may be positioned in the knee component 12, a pylon component, and/or a prosthetic foot component of the lower limb prosthetic device 10. In some aspects, a first sensor 104A may be positioned posterior to the longitudinal axis 16 and a second sensor 104B may be positioned anterior to the longitudinal axis 16, as shown in FIG. 1B.

In some aspects, the control system 100 may include a microcontroller 106. In some aspects, the IMU 102 may include the microcontroller 106 so the microcontroller 106 is integrated into (i.e., part of) the IMU 102. In some aspects, the microcontroller 106 may be coupled to or connected to the IMU 102 and/or the sensors 104. The microcontroller 106 may be coupled to or connected to the IMU 102 and/or the sensors 104 via a wired or wireless connections. The IMU 102 and/or the sensors 104 may transmit data or information to the microcontroller 106. The microcontroller 106 may receive the data or information from the IMU 102 and/or the sensors 104. In some aspects, the microcontroller 106 may transmit data or information from the IMU 102 and/or the sensors 104. The IMU 102 may transmit to the microcontroller 106 data or information representative of the acceleration determined or measured by the accelerometer 102A and/or data or information representative of the angular velocity determined or measured by the gyroscope 102B. The sensors 104 may transmit data or information representative of the forces measured or determined by the sensors 104 to the microcontroller 106. The microcontroller 106 may calculate or determine information associated with the lower limb prosthetic device 10. The information associated with the lower limb prosthetic device 10 may include one or more of a shank angle of the lower limb prosthetic device 10, a velocity of the lower limb prosthetic device 10, a position of the lower limb prosthetic device 10, a type or terrain of the walking surface and/or any other information associated with the lower limb prosthetic device 10 based at least in part on the data or information received by the microcontroller 106 from the IMU 102 and/or the sensors 104. The microcontroller 106 may generate instructions based at least in part on the data or information received by the microcontroller 106 from the IM 102 and/or the sensors 104, and/or the information associated with the lower limb lower limb prosthetic device 10. The microcontroller 106 may transmit the instructions to the one or more actuation systems 20 of the lower limb prosthetic device 10 to control the one or more actuation systems 20. The one or more actuation systems can affect movements of the lower limb prosthetic device 10 appropriately. The location of the actuation system 20, the IMU 102, the microcontroller 106, and/or the sensors 104 on the knee component 12 as illustrated in FIG. 1B are exemplary and not limiting.

FIG. 2A illustrates an example gait cycle 200. The gait cycle 200 may be defined between when a first foot 202 of a user 201 is in a specific position and the next time the first foot 202 in the same position. For example, the gait cycle 200 may be defined between when a heel end of the first foot 202 contacts a walking surface (that is, heel strike) at a first time and when the heel end of the first foot 202 contacts the walking surface a second time. The gait cycle 200 may include a stance phase 204 and a swing phase 206. The stance phase 204 may include a portion of the gait cycle 200 when at least a portion of the first foot 202 contacts the walking surface. The swing phase 206 may include a portion of the gait cycle 200 when the first foot 202 does not contact the walking surface. The stance phase 204 may include a double support state 210 and a single support state 212. The double support state 210 may include a portion of the stance phase 204 when the first foot 202 and a second foot 203 both contact the walking surface. The single support state 212 may include a portion of the stance phase 204 when the first foot 202 contacts the walking surface and the second foot 203 does not contact the walking surface.

FIG. 2B illustrates forces F detected or measured by the sensors 104 throughout a gait cycle 200. As shown, the forces F may be larger or increase when the first foot 202 of the user 201 is in contact with the walking surface between a loading response and a pre-swing (i.e., the single support state 212). As described further below, the control system 100 and/or the microcontroller 106 may use an increase in the forces F to determine when the user 201 is in a single support state 212.

FIG. 2C illustrates schematically a lower limb prosthetic device (for example, the lower limb prosthetic device 10 as shown in FIGS. 1A-1C) in the single support state 212. The lower limb prosthetic device may be represented or modeled as an inverse pendulum when the lower limb prosthetic device is in the single support state 212, as described further below. In some aspects, rotation of a shaft 18 of the lower limb prosthetic device may be represented or modeled as an inverse pendulum where the IM 102 is positioned a distance 220 from an ankle joint 22 of the lower limb prosthetic device. In some aspects, when the lower limb prosthetic device is in the single support state 212, the acceleration of the lower limb prosthetic device may be modeled or calculated using Equation 1 below:

$$a = L \frac{d ω}{d t} \qquad Equation 1$$

The acceleration (a) of the lower limb prosthetic device may be the product of the distance 220 (L) and the derivative of the angular velocity (o) over time (t).

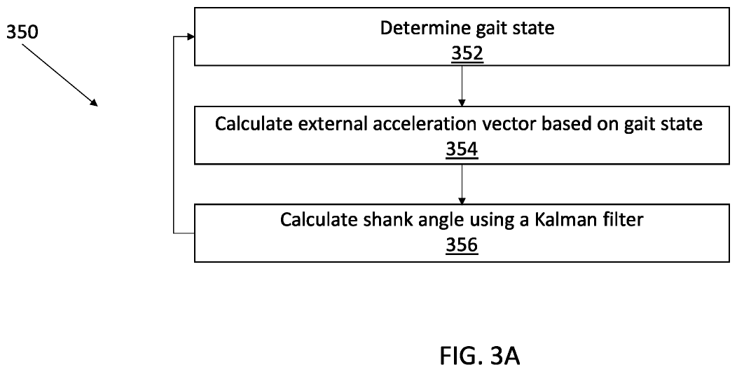

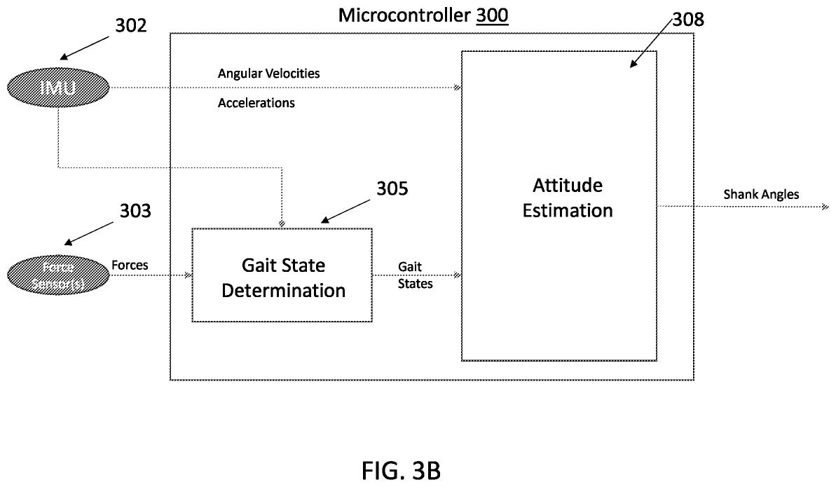

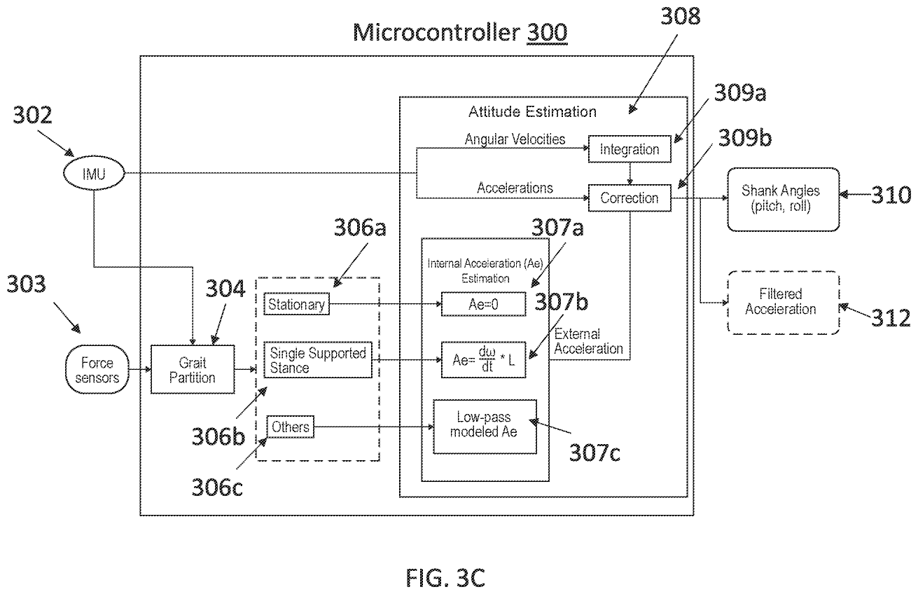

FIG. 3A illustrates an example method 350 for determining (e.g., tracking) a shank angle of the lower limb prosthetic device. In some aspects, the method 350 may reduce, minimize, or eliminate drift in the shank angle determined using measurements from an IMU. At step 352, the control system may determine the gait state of the user. The control system may determine the gait state of the lower limb prosthetic based at least in part of measurements from the IMU and/or the force sensors. At step 354, the control system can calculate an external acceleration vector based on the gait state of the user. At step 356, the control system may calculate the shank angle of the lower limb prosthetic. The control system may calculate the shank angle using the angular velocity and acceleration data from the IMU and the external acceleration vector. The control system may calculate the shank angle using a Kalman filter. The control system may use the angular velocity of the lower limb prosthetic as an input for a prediction step. The system may use the external acceleration vector and the acceleration of the lower limb prosthetic as inputs for a correction step of the Kalman filter. The control system may then return to step 352 to repeat method 350.

FIGS. 3B and 3C are block diagrams illustrating how the control system disclosed herein can determine the shank angle (see shank angle 250 in FIG. 2C) of the lower limb prosthetic device disclosed herein. The shank angle is an angle between the shank and a ground frame z-axis Z^{G} (i.e., a vertical axis). The vertical axis may be perpendicular to a ground that the prosthetic foot makes contact with. FIG. 3C is a more detailed block diagram of FIG. 3B illustrating certain non-limiting examples of an attitude estimation block of the control system. The blocks in the system diagrams in the present disclosure (e.g., FIGS. 3B-3C, 4B, and 5A) may include software and/or hardware processing units.

In some aspects, the control system may reduce, minimize or eliminate drift in the shank angle determination using measurements from an IMU 302, which may be the IMU 102 shown in FIGS. 1A-1B. In some aspects, a Kalman filter may be used by a microcontroller 300 for sensor fusion.

As shown in FIGS. 3B and 3C, the IMU 302 may collect data or information, which can be fed to various software and/or hardware blocks of the microcontroller 300 (which may be the microcontroller 106 of FIGS. 1A-1B). The IMU 302 may measure or determine the angular velocity and the acceleration of the lower limb prosthetic device. The angular velocities and accelerations data from the IMU 302 can be fed into an attitude estimation block 308 of the microcontroller 300. In addition, force sensors 303 (which may be the sensors 104 of FIGS. 1A and 1B) may measure or determine the force applied to the lower limb prosthetic device. The force measurements can be fed into a gait state determination block 305 of the microcontroller 300 as shown in FIG. 3B, or a gait partition block 304 of the microcontroller 300 as shown in FIG. 3C. Signals from the IMU 302 can also be fed into the gait state determination block 305 or the gait partition block 304. As also shown in FIG. 3B, the gait state determination block 305 can determine the gait state of the user (for example, using the methods disclosed herein based at least in part on the force sensor measurements or otherwise) and feed the gait state information to the attitude estimation block 308. The attitude estimation block 308 can determine the shank angles. The attitude estimation block 308 can determine an external acceleration component based on the gait state information, which can improve the accuracy in the shank angle determination. For example, the microcontroller 300 can fuse the data from the IMU 302 with the external acceleration component (e.g., by using a Kalman filter or otherwise) to estimate the shank angles. The shank angles may be estimated in pitch and roll. The control system disclosed herein can control the actuation systems of the lower limb prosthetic device based on the shank angles.

As shown in FIG. 3C, the attitude estimation block 308 of the microcontroller 300 can perform an integration 309a of the angular velocities data received from the IMU 302. The output of the integration 309a and the acceleration data from the IMU 302 can be fed into a correction block 309b. The microcontroller 300 can further determine an external acceleration component (described in more details below), which can also be fed into the correction block 309b. The correction block 309b can calculate and output shank angles 310 based at least in part on the inputs disclosed herein.

The external acceleration component can be determined at least in part based on the gait state of the lower limb prosthetic device. As shown in FIG. 3C, the gait partition block 304 of the microcontroller 300 may determine a gait state of the lower limb prosthetic device based on the sensor data from the IMU 302 and the force cells. Alternatively, the microcontroller 300 can determine the gait state using the shank angles. The gait partition block 304 may output a stationary state 306a if the lower limb prosthetic device is stationary. The gait partition block 304 may output a single supported stance state 306b if the lower limb prosthetic device is in the single support state of a gait cycle. The gait partition block 304 may output an “others” state 306c if the lower limb prosthetic device is neither stationary nor in the single support state. In some aspects, the lower limb prosthetic device may be stationary if one or more stationary determination conditions are met.

The attitude estimation block 308 of the microcontroller 300 can estimate an external acceleration (“Ae”) component (e.g., an Ae vector or otherwise) using different assumptions and/or models based on the output 306a, 306b, 306c of the gait partition block 304. With a stationary state output 306a, the microcontroller 300 can assume there is zero external acceleration 307a. With a single supported stance output 306b, the microcontroller 300 can calculate an external acceleration component using an inverted pendulum model 307b disclosed herein. With an “others” state output 306c, the microcontroller 300 can calculate an external acceleration component using a low pass filtered model 307c disclosed herein.

More details of how each of the three states are determined by the microcontroller 300 will be described below. In some aspects, the one or more stationary determination conditions may include whether a norm value of the acceleration of the lower limb prosthetic device is within an acceleration threshold of gravitational acceleration (i.e., about 9.8 m/s²). The norm value of the acceleration may include a magnitude of a vector representation of the acceleration. The acceleration threshold may include about 0.01 m/s², about 0.02 m/s², about 0.03 m/s², about 0.04 m/s², about 0.05 m/s², about 0.06 m/s², about 0.07 m/s², about 0.08 m/s², about 0.09 m/s², about 0.10 m/s², about 0.11 m/s², about 0.12 m/s², about 0.13 m/s², about 0.14 m/s², about 0.15 m/s², about 0.20 m/s², and/or any value between the aforementioned values.

Additionally or alternatively, the one or more stationary determination conditions may include whether the norm value of the acceleration of the lower limb prosthetic device is within the acceleration threshold of gravitational acceleration for a predefined time threshold. The time threshold may include about 10 milliseconds (ms), about 20 ms, about 30 ms, about 40 ms, about 50 ms, about 60 ms, about 70 ms, about 80 ms, about 90 ms, about 100 ms, about 110 ms, about 120 ms, about 130 ms, about 140 ms, about 150 ms, about 160 ms, about 170 ms, about 180 ms, about 190 ms, about 200 ms, and/or any value between the aforementioned values.

Additionally or alternatively, the one or more stationary determination conditions may include whether the norm value of the acceleration of the lower limb prosthetic device is within the acceleration threshold of gravitational acceleration for a predefined number of measurements. The number of measurements may include 1 measurement, 2 measurements, 3 measurements, 4 measurements, 5 measurements, 6 measurements, 7 measurements, 8 measurements, 9 measurements, 10 measurements, 11 measurements, 12 measurements, 13 measurements, 14 measurements, 15 measurements, 16 measurements, 17 measurements, 18 measurements, 19 measurements, and/or 20 measurements.

Additionally or alternatively, the one or more stationary determination conditions may include whether a change in the angular velocity of the lower limb prosthetic device in the direction of one or more axes of the body-fixed frame F^{S} is less than an angular velocity threshold. In some aspects, the one or more stationary determination conditions may include whether a change in the angular velocity of the lower limb prosthetic device in the direction of every axis of the body-fixed frame F^{S} is less than the angular velocity threshold. In some aspects, the angular velocity threshold may include about 0.01 rad/s, about 0.02 rad/s, about 0.03 rad/s, about 0.04 rad/s, about 0.05 rad/s, about 0.06 rad/s, about 0.07 rad/s, about 0.08 rad/s, about 0.09 rad/s, about 0.10 rad/s, and/or any value between the aforementioned values. In some aspects, the one or more stationary determination conditions may include whether the angular velocity of the lower limb prosthetic device is less than the angular velocity threshold for a predefined time threshold. In some aspects, the one or more stationary determination conditions may include whether the angular velocity of the lower limb prosthetic device is less than the angular velocity threshold for a predefined number of measurements.

Additionally or alternatively, the lower limb prosthetic device may be in the single supported stance state when the lower limb prosthetic device is not stationary (i.e., the one or more stationary determination conditions are not met). In some aspects, the lower limb prosthetic device may be in the single supported stance state when the lower limb prosthetic device is not stationary and one or more single support conditions are met.

Additionally or alternatively, the one or more single supported stance state conditions may include whether a sum of the forces applied to each sensor 303 is larger than a single support force threshold. In some aspects, the single support force threshold may include a force of about 10 Newtons (N), 50 N, about 100 N, about 150 N, about 200 N, about 250 N, about 300 N, about 350 N, about 400 N, about 450 N, about 500 N, about 550 N, about 600 N, about 650 N, about 700 N, about 750 N, about 800 N, about 850 N, about 900 N, about 950 N, about 1000 N, about 1050 N, about 1100 N, about 1150 N, about 1200 N, about 1250 N, about 1300 N, about 1350 N, about 1400 N, about 1450 N, about 1500 N, and/or any value between the aforementioned values.

Additionally or alternatively, the one or more single supported stance state conditions may include whether a difference between the norm value of the acceleration of the lower limb prosthetic device and gravitational acceleration is less than a first single support acceleration threshold. In some aspects, the first single support acceleration threshold may include an acceleration of about 0.5 m/s², about 1 m/s², about 1.5 m/s², about 2 m/s², about 2.5 m/s², about 3 m/s², about 3.5 m/s², about 4 m/s², about 4.5 m/s², about 5 m/s², about 5.5 m/s², about 6 m/s², about 6.5 m/s², about 7 m/s², about 7.5 m/s², about 8 m/s², about 8.5 m/s², about 9 m/s², about 9.5 m/s², and/or any value between the aforementioned values.

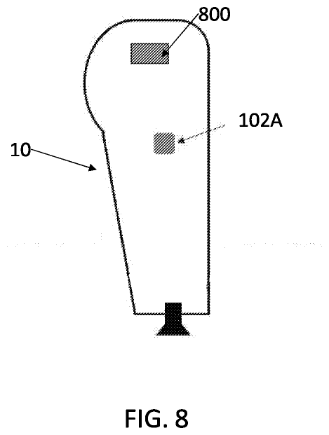

Additionally or alternatively, the one or more single supported stance state conditions may include using data from a knee angle sensor 800 and the accelerometer 102A, as shown in FIG. 8. The locations of the knee angle sensor 800 and/or the accelerometer 102A as shown in FIG. 8 are for illustrative purposes and not limiting. The knee angle sensor 800 and/or the accelerometer 102A can be placed at any suitable location on the lower limb prosthetic device 10. Data from the knee angle sensor 800 and the accelerometer 102A can be used to convert to an inverted pendulum model (described in more details elsewhere in the present disclosure with reference to FIG. 2C). The one or more single supported stance state conditions can be determined partially based on whether a knee joint of the lower limb prosthetic is fully extended. In some aspects, the knee joint of the lower limb prosthetic may include the knee angle sensor 800. In some aspects, the knee angle sensor 800 may include a rotational hall sensor. The knee angle sensor 800 may measure a joint angle of the knee joint. The joint angle may include an angle when the knee joint is bent. In some aspects, the knee joint of the lower limb prosthetic may be fully extended when the joint angle of the knee joint is about 0 degrees. In some aspects, the knee joint of the lower limb prosthetic may be fully extended when the joint angle of the knee joint is less than a fully extended threshold angle. In some aspects, the fully extended threshold angle may include an angle of about 0.5 degrees, about 1 degree, about 1.5 degrees, about 2 degrees, about 2.5 degrees, about 3 degrees, about 3.5 degrees, about 4 degrees, about 4.5 degrees, about 5 degrees, about 6 degrees, about 7 degrees, about 8 degrees, about 9 degrees, about 10 degrees, and/or any value between the aforementioned values. In one example, the knee joint angle measured by the knee angle sensor 800 indicates that the knee is fully extended, the single supported state can be assumed when the accelerometer data is equal to gravity for a predetermined duration. In some aspects, the predetermined duration may include about 5 milliseconds (ms), about 10 ms, about 20 ms, about 30 ms, about 40 ms, about 50 ms, about 60 ms, about 70 ms, and/or any value between the aforementioned values. In some aspects, the knee angle sensor 800 can replace the force sensors 104, 303 in the examples disclosed herein.

Additionally or alternatively, the one or more single supported stance state conditions may include whether a number of previous external acceleration estimates are less than a second single support acceleration threshold. A previous external acceleration estimate may include an external acceleration estimate generated when the shank angle was estimated at a previous time. For example, if the microcontroller is estimating the shank angle for a third time and the number of previous external acceleration estimates is two (2), the previous external acceleration estimates may include the previous external acceleration estimate generated at the second time and at the first time.

In some aspects, the number of previous external acceleration estimates may include one (1) previous external acceleration estimate, two (2) previous external acceleration estimates, three (3) previous external acceleration estimates, four (4) previous external acceleration estimates, five (5) previous external acceleration estimates, six (6) previous external acceleration estimates, seven (7) previous external acceleration estimates, eight (8) previous external acceleration estimates, nine (9) previous external acceleration estimates, ten (10) previous external acceleration estimates, twenty (20) previous external acceleration estimates, thirty (30) previous external acceleration estimates, forty (40) previous external acceleration estimates, fifty (50) previous external acceleration estimates, or more, or any value between the aforementioned values.

In some aspects, the second single support acceleration threshold may include an acceleration of about 0.5 m/s², about 1 m/s², about 1.5 m/s², about 2 m/s², about 2.5 m/s², about 3 m/s², about 3.5 m/s², about 4 m/s², about 4.5 m/s², about 5 m/s², about 5.5 m/s², about 6 m/s², about 6.5 m/s², about 7 m/s², about 7.5 m/s², about 8 m/s², about 8.5 m/s², about 9 m/s², about 9.5 m/s², and/or any value between the aforementioned values.

In some aspects, the lower limb prosthetic device may be in a portion of the gait cycle 200 other than the single support state when the lower limb prosthetic device is neither stationary nor in the single support state.

As described above, the attitude estimation block 308 of the microcontroller 300 may generate or calculate an external acceleration vector based at least in part on the gait state of the lower limb prosthetic device. The external acceleration vector may be about zero (0) m/s^{s} in the direction of the shaft x-axis X^{S} and the direction of the shaft y-axis Y^{S}, and gravitational acceleration in the direction of the shaft z-axis Z^{S} when the microcontroller 300 determines that the lower limb prosthetic device is stationary. The attitude estimation block 308 may calculate an external acceleration vector using Equation 1 disclosed herein with reference to FIG. 2C (that is the inverted pendulum model), when the microcontroller 300 determines that the lower limb prosthetic device is in the single supported stance state. Alternatively, the stationary state and single supported stance phase can be combined as one condition in which the inverted pendulum model is used.

The attitude estimation block 308 may calculate an external acceleration vector using a low pass filter model when the microcontroller 300 determine that the lower limb prosthetic device is neither stationary nor in the single supported stance state. In some aspects, the low pass filter model may include applying Equation 2 below:

$${a}_{t}^{-} = {c}_{a} {a}_{t - 1} \qquad Equation 2$$

The external acceleration vector

$$( {a}_{t}^{-} )$$

may be a product of a constant (cₐ) and a filtered acceleration vector determined at a previous time(aₜ₋₁). The constant (cₐ) may include a dimensionless constant that may determine a cutoff frequency of the low pass filter. The constant (cₐ) may include a value between about 0.0 and 1.0. In some aspects, the constant (cₐ) may include a value of about 0.1, about 0.2, about 0.3, about 0.4, about 0.5, about 0.6, about 0.7, about 0.8, about 0.9, about 0.99, and/or any value between the aforementioned values.

More details of how the attitude estimation block 308 of the microcontroller 300 may determine or calculate the shank angle using the angular velocities and accelerations data from the IMU 302 and the external acceleration vector will now be described. The attitude estimation block 308 can determine a last row of a Direction Cosine Matrix (DCM) between the body-fixed frame F^{S} of the lower limb prosthetic device 10 and a ground frame F^{G} (i.e., stride reference frame) (show in FIG. 2C). The DCM is one way to represent the attitude of a rigid body in a three dimensional space. Any vector V in the ground frame F^{G} (see FIG. 2C) generates three angles with the body-fixed frame F^{S}. The cosines of the angles between the vector and the base coordinate frame are represented by direction cosines. The last row of the DCM may include elements that are dependent on a pitch angle and roll angle. Therefore, the attitude estimation may be determined or calculate based on the last row of the DCM. The microcontroller 300 may use a Kalman filter to calculate the last row of the DCM. The microcontroller 300 may use the angular velocity of lower limb prosthetic device determined or measured by the IMU 302 as an input for a prediction step of the Kalman filter. The microcontroller 300 may use the external acceleration vector and the acceleration as inputs for a correction step of the Kalman Filter.

In some aspects, the microcontroller 300 may use the angular velocity of lower limb prosthetic device as an input to determine the prediction of the last row of the DCM. The microcontroller 300 may integrate the angular velocity of the lower limb prosthetic device to determine the prediction of the last row of the DCM. The microcontroller 300 may use the external acceleration vector and the acceleration as inputs to correct the prediction of the last row of the DCM and generate a filtered last row of the DCM.

The microcontroller 300 may use the filtered last row of the DCM to determine or calculate the shank angle 310. In some aspects, the microcontroller 300 may set the z-axis (yaw angle) shank angle of the lower limb prosthetic device to about zero (0) degrees. In some aspects, the microcontroller 300 may use Euler Angles to determine the x-axis shank angle (i.e., pitch angle) of the lower limb prosthetic device and/or the y-axis (i.e., roll angle) shank angle of the lower limb prosthetic 10.

In some aspects, the ground frame x-axis X^{G} shank angle (i.e., pitch angle) of the lower limb prosthetic device may be calculated using Equation 3 below:

$$γ = arctan ( \frac{{}^{B} {Z}_{2}}{{}^{B} {Z}_{3}} ) \qquad Equation 3$$

The ground frame x-axis X^{G} shank angle (γ) may be an inverse tangent of a quotient of a second element in the last row of the DCM (^{B}Z₂) and a third element in the last row of the DCM (^{B}Z₃).

In some aspects, the ground frame y-axis Y^{G} (i.e., roll angle) shank angle of the lower limb prosthetic device may be calculated using Equation 4 below:

$$β = arctan ( \frac{- {}^{B} {Z}_{1}}{\sqrt{{}^{B} {Z}_{2}^{2} + {}^{B} {Z}_{3}^{2}}} ) \qquad Equation 4$$

The ground frame y-axis Y^{G} shank angle (β) may be an inverse tangent of a quotient of a negation of a first element in the last row of the DCM (^{B}Z₁) and a square root of a sum of a square of the second element in the last row of the DCM (^{B}Z₂) and a square of the third element in the last row of the DCM (^{B}Z₃).

Optionally, the microcontroller 300 may determine or calculate a filtered acceleration vector 312. In some aspects, the filtered acceleration vector may be calculated using Equation 5 below:

^{B}a=yₐ−g×^{B}Z Equation 5

The filtered acceleration vector (^{B}a) 312 may be a vector representation of the acceleration determined or measured by the IMU 302 (yₐ) minus a product of a gravitational acceleration vector (g) and the last row of the DCM (^{B}Z).

In some aspects, the control system of the lower limb prosthetic device disclosed herein may periodically repeat the shank angle determinations as shown in FIGS. 3A and 3B. The control system may repeat the shank angle determination each time the control system collects data or information from the sensors (e.g., the IMU and/or force sensors, etc.). In some aspects, the control system may periodically collect data or information at a sample rate. In some aspects, the sample rate may include a frequency of about 1 Hz, about 5 Hz, about 10 Hz, about 20 Hz, 30 Hz, about 40 Hz, about 50 Hz, about 60 Hz, about 70 Hz, about 80 Hz, about 90 Hz, about 100 Hz, about 110 Hz, about 120 Hz, about 130 Hz, about 140 Hz, about 150 Hz, about 160 Hz, about 170 Hz, about 180 Hz, about 190 Hz, about 200 Hz, about 210 Hz, about 220 Hz, about 230 Hz, about 240 Hz, about 250 Hz, about 260 Hz, about 270 Hz, about 280 Hz, about 290 Hz, about 300 Hz, about 350 Hz, about 400 Hz, about 450 Hz, about 500 Hz, about 550 Hz, about 600 Hz, about 650 Hz, about 700 Hz, about 750 Hz, about 800 Hz, about 850 Hz, about 900 Hz, about 950 Hz, about 1000 Hz, and/or any value between the aforementioned values.

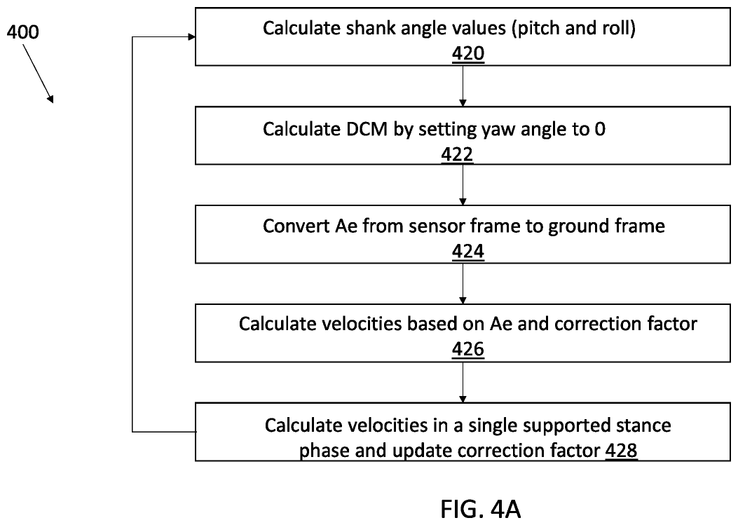

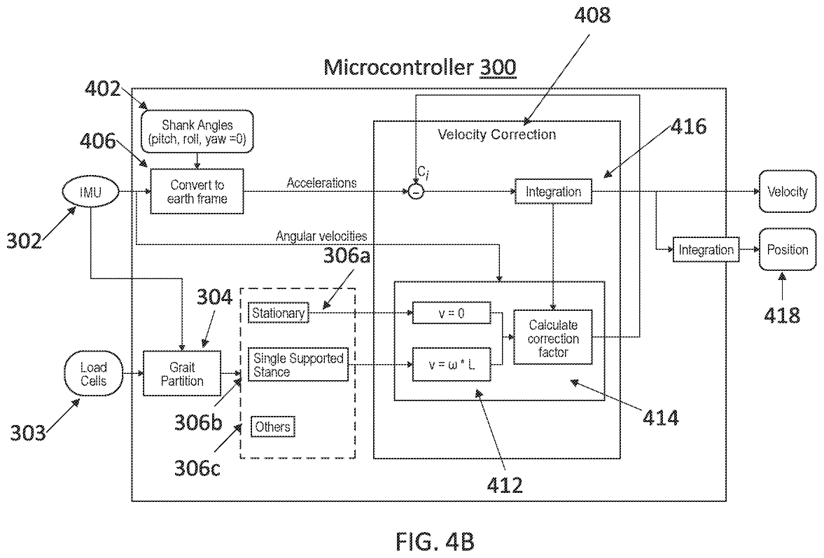

The shank angles determined based on the block diagrams in FIGS. 3A and 3B are in pitch and roll. More details of the velocity and/or position determination by the control system after the shank angles in pitch and roll and the external acceleration of the lower limb prosthetic device are obtained will be described below with reference to FIGS. 4A and 4B. The shank angles in sagittal plane (pitch) and frontal plane (roll), and external accelerations of the lower limb prosthetic device may also collectively be referred to as the attitude of the lower limb device. The velocity and/or position determination disclosed herein can be based in part on the attitude determination and/or the gait information.

FIG. 4A illustrates an example method 400 for determining a velocity and/or position of the lower limb prosthetic device. In some aspects, the method 400 may reduce, minimize, or eliminate drift in the velocity and/or position determined using measurements from an IMU 102. At step 420, the control system can calculate the shank angles in pitch and roll (for example, by following the block diagrams in FIG. 3A or 3B, or otherwise). At step 422, the control system can set the yaw angle to zero and calculate the DCM using the shank angles in pitch, yaw, and roll. The yaw angle can be set to zero because the heading direction (i.e., the yaw angle)'s orientation does not hold significance in the velocity and/or position determination process. At step 424, the control system can convert the external acceleration vector (for example, by following the block diagrams in FIG. 3A or 3B, or otherwise) from a sensor frame to a ground frame. At step 424, the control system can calculate the velocities of the lower limb prosthetic device based on the converted external acceleration vector and a velocity correction factor. The determination of the velocity correction factor will be described in greater detail below with reference to FIG. 4B. In some aspects, the control system can calculate the velocities by integrating the converted external acceleration vector with the velocity correction factor. During the swing phase, the velocity can be determined through the integration of the acceleration data from the IMU. However, since this process integrates both actual accelerations and errors, it leads to the generation of drifts that progressively escalate without subsequent correction. When it comes to the stance phase, particularly during the single supported stance phase, a more accurate velocity can be acquired from the inverted pendulum model disclosed herein. At step 428, the control system can calculate the velocities of the lower limb prosthetic device in a single supported stance phase, which can be used to update the velocity correction factor. The control system can adjust and correct the velocity that includes the drift in the next gait cycle using the updated correction factor. The control system can also determine the position of the lower limb prosthetic device by integrating the corrected velocity. The control system can then return to step 420 to repeat the method 400.

FIG. 4B illustrates an example block diagram illustrating how the control system can determine a velocity and/or position of the lower limb prosthetic device.

For velocity and/or position determination, the microcontroller 300 of the lower limb prosthetic device may collect data or information from the IMU 302 and/or the force sensors 303. In some aspects, the force sensors 303 can be load cells. In some aspects, the microcontroller 300 may receive the angular velocities data of the lower limb prosthetic device, the acceleration data of the lower limb prosthetic device, and/or forces applied to the lower limb prosthetic device. In some aspects, the IMU 302 may measure or determine the angular velocity and the acceleration of the lower limb prosthetic device. In some aspects, the force sensors 303 may measure or determine the forces applied to the lower limb prosthetic device. Additionally, the microcontroller 300 can determine the shank angles in pitch and roll.

Additionally, the microcontroller 300 may determine the shank angle of the lower limb prosthetic device. In some aspects, the microcontroller 300 may determine the shank angle of the lower limb prosthetic device by following the block diagrams in FIG. 3B or 3C, or otherwise. In some aspects, the microcontroller 300 may set the z-axis shank angle of the shank angle (the yaw angle) to zero (0) degrees.

The microcontroller 300 may determine the DCM based at least in part on the shank angles in pitch, yaw, and roll 402. At a converter block 406, the DCM can convert the external acceleration vector (for example, as determined by following the block diagram in FIG. 3B or 3C) from a sensor or body-fixed frame F^{S} of the lower limb prosthetic device to a ground frame F^{G}. In some aspects, the DCM may be calculate using Equation 6 below:

$$R = [ \begin{matrix}cos β cos α & sin γ sin β cos α - cos γ sin α & cos γ sin β cos α + sin γ sin α \\ cos β sin α & sin γ sin β sin α + cos γ cos α & cos γ sin β sin α - sin γ cos α \\ - sin β & sin γ cos β & cos γ cos β\end{matrix} ] \qquad Equation 6$$

The converter block 406 of the microcontroller 300 may output the acceleration of the lower limb prosthetic device in the ground frame F^{G}. The ground frame acceleration may be a product of the DCM and the acceleration of the lower limb prosthetic device as measured by the IMU 302. In some aspects, the acceleration may be the filtered acceleration vector determined by removing the external acceleration vector as disclosed herein. As also shown in FIG. 4B, the IMU 302 can output angular velocities data to a velocity correction block 408 of the microcontroller 300 for calculating a velocity correction factor ci in a subblock 414.

The microcontroller 300 may also use gait state information for calculating the velocity correction factor in the subblock 414. The microcontroller 300 may determine the gait state of the lower limb prosthetic device as disclosed herein, for example, using the gait partition block 304 as shown in FIG. 3C to output a stationary status 306a, a single supported stance status 306b, or an “others” status 306c.

With a stationary status output 306a, the ground frame velocity estimate of the lower limb prosthetic device may be zero (0) m/s². With a single supported stance status output state 306b, a body-fixed frame velocity vector may be calculated using Equation 7 below, where ω denotes the angular velocity vector and ωₓ, ω_{y} are two elements of ω representing the angular velocities about X-axis and Y-axis, respectively:

$${}^{B} {V}_{stance} = [ \begin{matrix}{}^{B} {v}_{x} \\ {}^{B} {v}_{y} \\ {}^{B} {v}_{z}\end{matrix} ] = f ( ω ) = L [ \begin{matrix}{ω}_{y} \\ - {ω}_{x} \\ 0\end{matrix} ] \qquad Equation 7$$

The body-fixed frame velocity estimate (^{B}v_{stance}) may be a product of the distance L (see FIG. 2C, 220) from the ankle joint of the lower limb prosthetic device to the IMU and the angular velocity vector. The body-fixed frame velocity vector (^{B}v_{stance}) may be converted to the ground frame velocity estimate by using Equation 8 below:

^{G}v_{stance}=R^{B}v_{stance} Equation 8

As disclosed elsewhere in the present disclosure, the ground frame velocity vector (^{G}v_{stance}) may be a product of the DCM (R) and the body-fixed frame velocity vector (^{B}v_{stance}).

The subblock 414 may determine or calculate the correction factor. The correction factor may be determined based at least in part on the gait state of the lower limb prosthetic. The correction factor may be determined based at least in part on the ground fame velocity vector.

With a stationary or single support state output, the correction factor may be calculated using Equation 9 below:

$${c}_{i} = {c}_{i - 1} + k \cdot \frac{Δ {v}_{i}}{{N}_{i}} \qquad Equation 9$$

The correction factor (cᵢ) may be a sum of a correction factor calculated for a previous gait cycle (cᵢ₋₁) and a product of a constant (k) and a quotient of a velocity difference (Δvᵢ) and a sample number (Nᵢ). In some aspects, the constant (k) may include a value between about 0 and about 1. In some aspects, the constant (k) may include a value of about 0.1, about 0.2, about 0.3, about 0.4, about 0.5, about 0.6, about 0.7, about 0.8, about 0.9, about 0.95, about 0.99, and/or any value between the aforementioned values. In some aspects, the sample number (Nᵢ) may include a number of times the microcontroller has determined shank angles in pitch and roll and/or the velocity and position when the lower limb prosthetic device is stationary and/or in a current single supported stance state.

The velocity difference (Δvᵢ) may include a difference between a ground frame velocity vector of the lower limb prosthetic device determined or calculated a previous time by integrating 416 the acceleration data or filtered acceleration data (i.e. using Equation 10, as described below), and the ground frame velocity vector of the lower limb prosthetic device determined or calculated based on the gait state. In some aspects, the velocity difference (Δvᵢ) may include the velocity difference (Δvᵢ) at the start of the single supported stance state 212. In some aspects, the velocity difference (Δvᵢ) may be updated each time the microcontroller 300 determines the velocity of the lower limb prosthetic device when the lower limb prosthetic device is stationary and/or in single supported stance state.

In some aspects, the correction factor may be an average every correction factor (cᵢ) calculated using Equation 9 during when the lower limb prosthetic device is stationary and/or in the single supported stance state.

In some aspects, if the microcontroller 300 determines that the lower limb prosthetic device is in an “others” state 306c, the correction factor may include the correction factor calculated using Equation 7 at the end of a previous single supported stance state. In some aspects, the correction factor may be initially set to zero (0). Therefore, if there is no previous single supported stance state, the correction factor may be zero (0) m/s² until the microcontroller 300 calculates a correction factor using Equation 9.

The microcontroller 300 may determine or calculate the velocity of the lower limb prosthetic device using integration 416. The integration 416 can be performed with the external acceleration and the correction factor. In some aspects, the integration 416 may use Equation 10 below:

v=∫(a−cᵢ)dt Equation 10

The velocity (v) of the lower limb prosthetic device may be an integral over time (dt) of the ground frame acceleration (a) minus the correction factor (cᵢ) determined at subblock 414. In some aspects, as described above, the microcontroller 300 may use the velocity (v) of the lower limb prosthetic device to updated the velocity difference (Δvᵢ) at subblock 414 the next time the microcontroller 300 determines the velocity of the lower limb prosthetic device. In other words, the correction factor can be updated using Equation 9.

As also shown in FIG. 4B, the microcontroller 300 may determine or calculate a position of the lower limb prosthetic device. In some aspects, the microcontroller 300 may determine or calculate the position of the 10 based at least in part on the velocity of the lower limb prosthetic device determined or calculated by integration 416. In some aspects, the position of the lower limb prosthetic device may be an integral over time of the velocity of the lower limb prosthetic device.

In some aspects, the control system may periodically determine the velocity and/or position of the lower limb prosthetic device, for example, by repeating method 400 in FIG. 4A or following the block diagram in FIG. 4B. The control system may repeat data or information collection from the IM 302 and/or the force sensors 303. In some aspects, the control system may periodically collect data or information from the IMU 302 and/or the force sensors 303 at a sample rate. In some aspects, the sample rate may include a frequency of about 1 Hz, about 5 Hz, about 10 Hz, about 20 Hz, 30 Hz, about 40 Hz, about 50 Hz, about 60 Hz, about 70 Hz, about 80 Hz, about 90 Hz, about 100 Hz, about 110 Hz, about 120 Hz, about 130 Hz, about 140 Hz, about 150 Hz, about 160 Hz, about 170 Hz, about 180 Hz, about 190 Hz, about 200 Hz, about 210 Hz, about 220 Hz, about 230 Hz, about 240 Hz, about 250 Hz, about 260 Hz, about 270 Hz, about 280 Hz, about 290 Hz, about 300 Hz, about 350 Hz, about 400 Hz, about 450 Hz, about 500 Hz, about 550 Hz, about 600 Hz, about 650 Hz, about 700 Hz, about 750 Hz, about 800 Hz, about 850 Hz, about 900 Hz, about 950 Hz, about 1000 Hz, and/or any value between the aforementioned values.

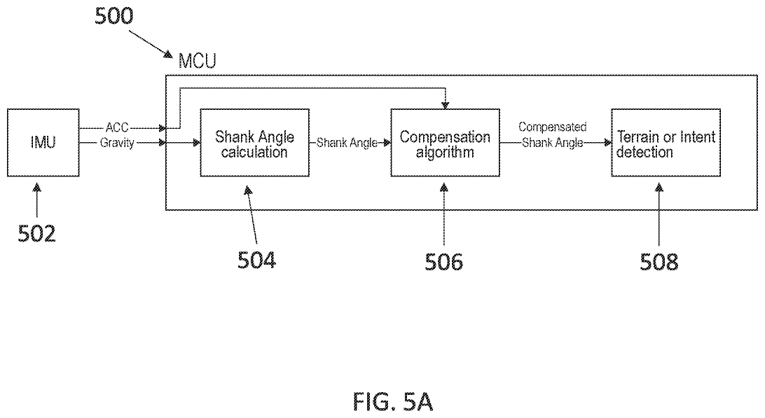

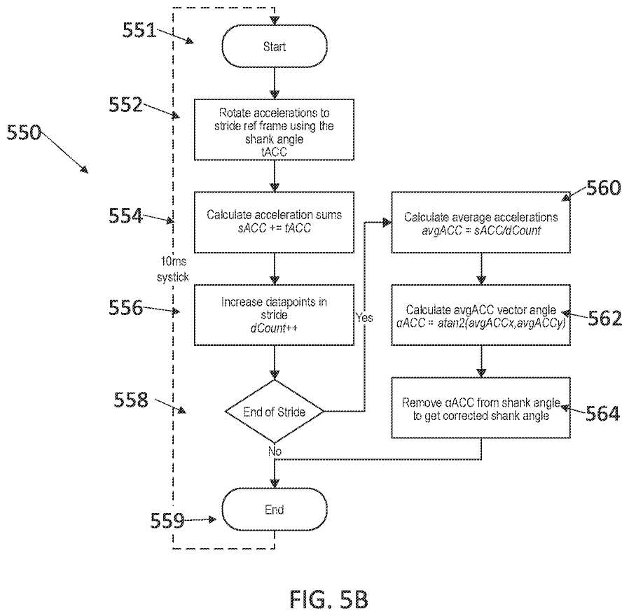

FIG. 5A illustrates a control system for reducing or minimizing drift in a shank angle estimation of the lower limb prosthetic device disclosed herein. As disclosed elsewhere in the present disclosure, the control system can perform orientation estimations (with shank angle being one of the non-limiting examples), for example, by integrating the IM measurements to get orientation angles. The orientation estimations tend to drift as the measured accelerations and angular velocities are noisy signals. The noise can be minimized by calibrating the sensor, but the calibration may not remove all the noises in the IMU signals, which are enlarged during integration. The drift in shank angle may be into both the positive and negative directions. The control system may implement a compensation algorithm, which may include calculating the average acceleration in the stride reference frame and the vector angle. The vector angle of the average accelerations correlates well with the drift described herein, as it is the shank angle that rotates the accelerations into the stride reference frame. Therefore, subtracting the vector angle from the shank angle can result in a drift compensated shank angle. More details of the compensation algorithm will be describe with reference to FIG. 5A. In some aspects, the control system may reduce, minimize or eliminate drift in the shank angle determined using measurement from an IMU. The control system may be the control system 100 illustrated in FIGS. 1A-1C, and/or the control systems illustrated in FIG. 3A, 3B or 4B. Accordingly, the control system of a lower limb prosthetic device can incorporate any of the drift reduction features disclosed herein.

The microcontroller 500 of the control system may receive a sample of data or information from the IMU 502. The data or information from the IMU 502 can include acceleration data and a gravity vector. In some aspects, the acceleration data can be in the sagittal plane. The gravity vector may be a vector representation of the acceleration of gravity in the body-fixed frame F^{S}. The acceleration data and the gravity vector can be fed into a shank angle calculation block 504 of the microcontroller 500.

The shank angle calculation block 504 of the microcontroller 500 may determine or calculate the shank angle of the lower limb prosthetic device. In some aspects, the microcontroller 500 may determine or calculate the shank angle in a sagittal plane (i.e., the ground frame x-axis X^{G} shank angle). In some aspects, the microcontroller 500 may determine or calculate the shank angle as described herein with reference to FIGS. 3A and 3B or otherwise. In some aspects, the microcontroller 500 may use the gravity vector and the acceleration data to determine or calculate the shank angle. In some aspects, the shank angle may be calculated using Equation 11 below:

γ=atan2(G_{y},G_{z}) Equation 11

The shank angle in a sagittal plane (γ) may be the 2-argument arctangent of a value of the gravity vector in the direction of the shaft y-axis Y^{S} (G_{y}) and a value of the gravity vector in the direction of the shaft z-axis Z^{S} (G_{z}). The shank angle determined by the shank angle calculation block 504 and the acceleration data can be fed into a compensation block 506.

The compensation block 506 may generate a compensated shank angle. In some aspects, the compensation block 506 may use method 550, shown in FIG. 5B, to determine or calculate the compensated shank angle. In some aspects, the shank angle in the sagittal plane and an acceleration measured or determined by the IMU 502 may be used as inputs for the method 550. The compensated shank angle can be fed into a terrain or intent detection block 508 of the microcontroller to improve control of the lower limb prosthetic device.

In some aspects, the microcontroller 500 may implement the compensation algorithm each time the microcontroller 500 collects data or information from the IMU 502. In some aspects, the microcontroller 500 may periodically collect data or information from the IMU 502 at a sample rate. In some aspects, the sample rate may include a frequency of about 1 Hz, about 5 Hz, about 10 Hz, about 20 Hz, 30 Hz, about 40 Hz, about 50 Hz, about 60 Hz, about 70 Hz, about 80 Hz, about 90 Hz, about 100 Hz, about 110 Hz, about 120 Hz, about 130 Hz, about 140 Hz, about 150 Hz, about 160 Hz, about 170 Hz, about 180 Hz, about 190 Hz, about 200 Hz, about 210 Hz, about 220 Hz, about 230 Hz, about 240 Hz, about 250 Hz, about 260 Hz, about 270 Hz, about 280 Hz, about 290 Hz, about 300 Hz, about 350 Hz, about 400 Hz, about 450 Hz, about 500 Hz, about 550 Hz, about 600 Hz, about 650 Hz, about 700 Hz, about 750 Hz, about 800 Hz, about 850 Hz, about 900 Hz, about 950 Hz, about 1000 Hz, and/or any value between the aforementioned values.

FIG. 5B illustrates an example method 550 for generating a compensated shank angle. In some aspects, a timer (e.g., the systick) can be started. In some aspects, the timer can be started when a microcontroller receives power and/or is turned on. In some aspects, the time can be started at step 558 as described further below. At the start 551 of each timer (e.g., a systick timer or otherwise), the control system can sample the IMU measurements. The IMU measurements sampled can include the accelerometer data and the gyroscope data. In some aspects, all six channels of the IMU (i.e., accelerometer axes x, y, z, and gyroscope axes x, y, z, also referred to as Ax, Ay, Az, Gx, Gy, Gz) can be sampled. The microcontroller can calculate the shank angle (i.e., the uncompensated shank angle), for example, as described above with reference to 504 of FIG. 5A. For example, the microcontroller can calculate the uncompensated shank angle using data in the gravity axes that form the sagittal plane, for example, Gy and Gz. The shaft angle can be calculate using Equation 11.

Steps 552-564 illustrate a compensation algorithm, which may be performed by the compensation block 506 of FIG. 5A. At step 552 of method 550, the microcontroller may generate a rotated acceleration by the uncompensated shank angles determined at the start 551. The rotated acceleration may be the acceleration measured or determined by the IMU rotated from the body-fixed or sensor frame F^{S} to the ground or stride reference frame F^{G}. For example, the accelerometer data in the y and z axes may be rotated to the stride reference frame. In some aspects, the microcontroller may generate the rotated acceleration using Equations 12 and 13 below:

RA_{y}=A_{z} sin(γ)+A_{y} cos(γ) Equation 12

RA_{z}=A_{z} cos(γ)−A_{y} sin(γ) Equation 13

The rotated acceleration in the direction of the ground frame y-axis Y^{G}(RA_{y}) may be a sum of the product of the acceleration in the direction of the shaft z-axis Z^{S} (A_{z}) and the sine of the shank angle in a sagittal plane (γ) and the product of the acceleration in the direction of the shaft y-axis Y^{S} (A_{y}) and the cosine of the shank angle in a sagittal plane (γ). The rotated acceleration in the direction of the ground frame z-axis Z^{G}(RA_{z}) may be a sum of the product of the acceleration in the direction of the shaft z-axis Z^{S} (A_{z}) and the cosine of the shank angle in a sagittal plane (γ) and the product of the acceleration in the direction of the shaft y-axis Y^{S} (A_{y}) and the sine of the shank angle in a sagittal plane (γ).

At step 554, the microcontroller may generate a sum of the rotated acceleration signals. The sum may include a sum of each rotated acceleration in a particular axis generated each time the microcontroller performs step 552 of the method 550. In some aspects, the microcontroller may generate a y-axis acceleration sum (sACCy+=tACCy) and/or a z-axis acceleration sum (sACCz+=tACCz). In some aspects, at the start of a gait cycle, the acceleration sum may be set to zero (0) m/s².

At step 556, the microcontroller may increment a sample counter (dCount) by the number of samples used in step 554. In some aspects, the sample counter may be set to zero (0) at the start of a new stride. In some aspects, the sample counter may include a total number of samples used in calculating the sum. The microcontroller can use the updated number of samples to later calculate an average value.

At step 558, the microcontroller may determine whether an end of a stride is detected. In some aspects, the microcontroller may determine an end of a stride when the microcontroller determines that the lower limb prosthetic device is in a mid-stance position, a heel-strike position, and/or any other detectable gait event (such as shown in FIG. 2A). In some aspects, the microcontroller may detect a gait event using sensor data from the IMU and/or the other sensors (such as force sensors) as disclosed herein.

If at step 558, the microcontroller does not detect an end of a stride, at step 559 the microcontroller may return to the start 551 and repeat steps 552-556 until an end of a strike is detected. In some aspects, if no end of a strike is detected, a timer (e.g., a systick) can be started and the microcontroller may return to the start 551 and repeat steps 552-556 each time when the timer has expired. In some aspects, the timer can expire every 1 ms to 25 ms, or 10 ms to 20 ms, or 10 ms.

If at step 558, the microcontroller detects an end of a stride, the microcontroller can proceed to step 560. At step 560, the microcontroller may generate average acceleration values. In some aspects, the microcontroller may determine or calculate the average acceleration by dividing the acceleration sum in each rotated axis (e.g., the y and z axes) by the sample counter. The average acceleration in the rotated y axis can be calculated as avgACCy=sACCy/dCount. The average acceleration in the rotated z axis can be calculated as avgACCz=sACCz/dCount.

At step 562, the microcontroller may generate a vector angle for the average accelerations for the stride that has just ended. In some aspects, the microcontroller may determine or calculate the vector angle for the average acceleration in the direction of the ground frame y-axis Y^{G} (G_{y}) and with the average acceleration in the direction of the ground frame z-axis Z^{G} (G_{z}). The vector angle can be calculated as αACC=atan2(avgACCy, avgACCz). In some aspects, the vector angle may be converted from radians to degrees with αACC*π/180.

At step 564, the microcontroller may generate the compensated or corrected shank angle. The microcontroller may subtract the vector angle αACC from the shank angle to generate the compensated shank angle. In some aspects, when an end of a stride is detected and after step 560 is performed, the counter and/or the sum may be reset to zero. In some aspects, after computing the vector angle or the compensated shank angle, the microcontroller may set the sample counter and/or the sum to zero (0). The microcontroller may then proceed to step 559 to return to start 551. In some aspects, the microcontroller may return to start 551 after a systick timer has expired. With each iteration of the method 550, the microcontroller can compute a new compensated shank angle as the user moves the lower limb prosthetic device through gait cycles.

In some aspects, instead of detecting the end of a stride and calculating average accelerations for that stride, the microcontroller may calculate a moving average that spans more than one stride and then use the moving average at one or more gait events (e.g., heel strike, mid-stance, and/or the like) to calculate the vector angle. The microcontroller may perform a rolling compensated shank angle. In some aspects, at step 558, if the microcontroller determines the end of a stride has been detected and/or after the vector angle or the compensated shank angle has been computed, the microcontroller may not set the sample counter and/or the acceleration sum to zero (0). The microcontroller may remove an oldest rotated acceleration from the acceleration sum each time the microcontroller performs step 554 and decrement the counter by the number of samples from that oldest rotated acceleration.

While the shank angle compensation process is illustrated herein using two axes (the sagittal plane) of the gyroscope and the accelerometer as a non-limiting example, the same process can be performed using all three axes. It is to be appreciated one or more equations or steps of the methods described herein may be performed in one-axis, two-axes, and/or three-axis without departing from the scope of this disclosure. Additionally, any of the equations and the angles, velocities, accelerations, positions, and/or any other measurements or data described herein may include vector representations of the measurements or data in one-axis, two-axes, and/or three-axes without departing from the scope of this disclosure.

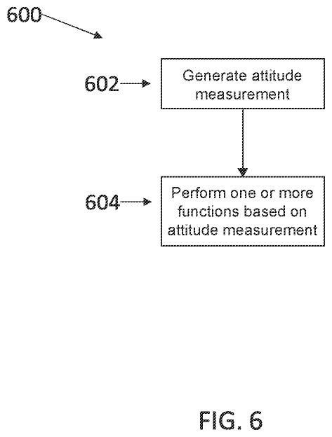

FIG. 6 illustrates a method 600 for controlling one or more functions of a lower limb prosthetic device disclosed herein. At step 602, the control system may generate an attitude measurement of the lower limb prosthetic device. In some aspects, the attitude measurement may include the shank angle determine or calculated using systems and methods disclosed herein. In some aspects, the attitude measurement may include the filtered acceleration vector determined or calculated using systems and methods disclosed herein. In some aspects, the attitude measurement may include the velocity or position of the lower limb prosthetic device determined or calculated using systems and methods disclosed herein. In some aspects, the attitude measurement may include the compensated shank angle determined or calculated using systems and methods disclosed herein.

At step 602, the control system may perform one or more functions based on the attitude measurement. In some aspects, the control system may generate instructions at least in part on the attitude measurement. In some aspects, the control system may transmit the instructions to the one or more actuation systems of the lower limb prosthetic device to control the one or more actuation systems. In some aspects, the control system may perform terrain detection, intent detection, and/or any other function, detection, or calculation based at least in part on the attitude measurement.

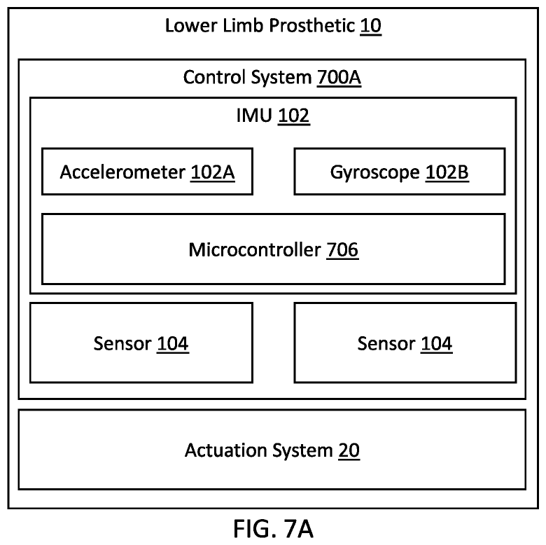

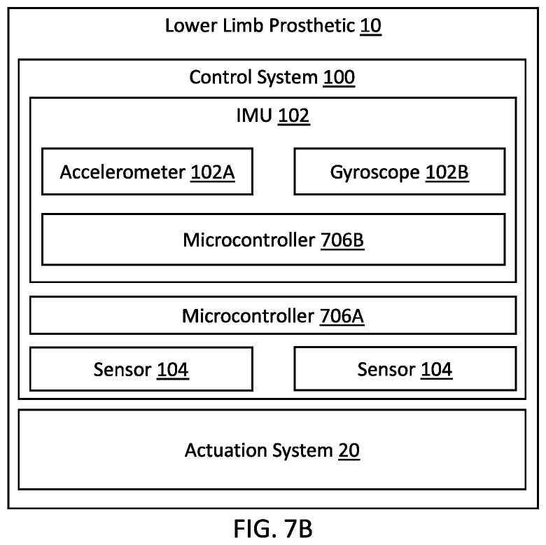

FIGS. 7A and 7B illustrate embodiments of the lower limb prosthetic device 10 with control system 700A and 700B. As shown in FIG. 7A, the IM 102 of the control system 700A may include the microcontroller 706. In some aspects, the microcontroller 706 may be integrated in the IM 102. As shown in FIG. 7B, the control system 700B may include a first microcontroller 706A and a second microcontroller 706B. In some aspects, the IMU 102 may include the second microcontroller 706B. In some aspects, the second microcontroller 706B may be integrated in the IMU 102.

It is to be appreciated that although the systems and/or methods 350, 400, 550, 600 described herein are described with reference to the control system 100 and/or the microcontroller 106, the control system 700A, 700B and/or microcontrollers 706, 706A, and/or 706B may be used to perform the systems and/or methods 350, 400, 550, 600.

Although this disclosure has been described in the context of certain embodiments and examples, it will be understood by those skilled in the art that the disclosure extends beyond the specifically disclosed embodiments to other alternative embodiments and/or uses and obvious modifications and equivalents thereof. In addition, while several variations of the embodiments of the disclosure have been shown and described in detail, other modifications, which are within the scope of this disclosure, will be readily apparent to those of skill in the art. It is also contemplated that various combinations or sub-combinations of the specific features and aspects of the embodiments may be made and still fall within the scope of the disclosure. For example, features described above in connection with one embodiment can be used with a different embodiment described herein and the combination still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the particular embodiments described above. Accordingly, unless otherwise stated, or unless clearly incompatible, each embodiment of this invention may comprise, additional to its essential features described herein, one or more features as described herein from each other embodiment of the invention disclosed herein.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

The various illustrative logical blocks, modules, and algorithm steps described in connection with the examples disclosed herein can be implemented as electronic hardware, computer software, or combinations of both. To clearly illustrate this interchangeability of hardware and software, various illustrative components, blocks, modules, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks and modules described in connection with the examples disclosed herein can be implemented or performed by a machine, such as a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be a controller, microcontroller, or state machine, combinations of the same, or the like. A processor can include electrical circuitry or digital logic circuitry configured to process computer-executable instructions. In another example, a processor can include an FPGA or other programmable device that performs logic operations without processing computer-executable instructions. A processor can also be implemented as a combination of computing devices, for example, a combination of a DSP and a microprocessor, a plurality of microprocessors, one or more microprocessors in conjunction with a DSP core, or any other such configuration. A computing environment can include any type of computer system, including, but not limited to, a computer system based on a microprocessor, a mainframe computer, a digital signal processor, a portable computing device, a device controller, or a computational engine within an appliance, to name a few.

The steps of a method, process, or algorithm described in connection with the examples disclosed herein can be embodied directly in hardware, in a software module stored in one or more memory devices and executed by one or more processors, or in a combination of the two. A software module can reside in RAM memory, flash memory, ROM memory, EPROM memory, EEPROM memory, registers, hard disk, a removable disk, a CD-ROM, or any other form of non-transitory computer-readable storage medium, media, or physical computer storage known in the art. An example storage medium can be coupled to the processor such that the processor can read information from, and write information to, the storage medium. In the alternative, the storage medium can be integral to the processor. The storage medium can be volatile or nonvolatile. The processor and the storage medium can reside in an ASIC.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise. Additionally, as used herein, “gradually” has its ordinary meaning (e.g., differs from a non-continuous, such as a step-like, change).

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A method for tracking a shank angle of a lower limb prosthetic device, the method comprising:
receiving sensor data from an accelerometer, a gyroscope, and at least a third sensor, wherein the third sensor comprises a plurality of force sensors, or a knee angle sensor;
determining a gait state of the lower limb prosthetic device based at least in part on the sensor data;
generating an external acceleration vector of a shank of the lower limb prosthetic based on the gait state of the lower limb prosthetic device; and
generating a shank angle vector of the shank based at least in part on the sensor data from the accelerometer and the gyroscope, and the external acceleration vector.

2. The method of claim 1, wherein the sensor data comprises an acceleration vector of the shank, and an angular velocity vector of the shank.

3. The method of claim 2, wherein the third sensor comprises the plurality of force sensors and the sensor data further comprises a force applied to the lower limb prosthetic device.

4. The method of claim 2, wherein the third sensor comprises the knee angle sensor and the sensor data further comprises a knee angle.

5. The method of claim 1, wherein a Kalman filter is used to generate the shank angle vector.

6. The method of claim 1, further comprising, controlling one or more functions of the lower limb prosthetic device based on the shank angle vector.

7. The method of claim 1, wherein the gait state comprises a stationary state, a single supported state or an others state.

8. The method of claim 7, wherein if the gait state is the stationary state, the external acceleration vector is zero, wherein if the gait state is the single supported state, the external acceleration vector is generated using an inverted pendulum model, and wherein if the gait state is the others state, the external acceleration vector is generated using a low pass filtered model.

9. The method claim 7, wherein the third sensor comprises the knee angle sensor, wherein the single supported state is assumed based on a knee angle indicative of a fully extended knee and accelerometer data being equal to gravity for a predetermined duration.

10. A method for tracking a shank angle of a lower limb prosthetic device, the method comprising:
receiving sensor data from an accelerometer and a gyroscope;
determining a shank angle of the shank based at least in part on the sensor data from the gyroscope;
generating a vector angle based on average acceleration values of a stride;
generating a compensated shank angle by subtracting the vector angle from the shank angle.

11. The method of claim 10, further comprising generating an acceleration sum vector of the stride.

12. The method of claim 11, further comprising generating average acceleration values of the stride based on the acceleration sum vector.

13. The method of claim 12, wherein average acceleration values of the stride are a quotient of the acceleration sum vector of the stride and a number of times sensor data is received during the stride.

14. The method of claim 11, wherein generating the acceleration sum vector comprises:
rotating an acceleration vector to a stride reference frame using the shank angle to generate a rotated acceleration vector;
adding the rotated acceleration vector to the acceleration sum vector.

15. (canceled)

16. A method for tracking a lower limb prosthetic device, the method comprising:
receiving sensor data from an accelerometer, a gyroscope, and at least a third sensor, wherein the third sensor comprises a plurality of force sensors or a knee angle sensor;
determining a shank angle of a shank of the lower limb prosthetic;
generating a converted external acceleration vector based on an external acceleration of the shank; and
generating a velocity of the lower limb prosthetic device based on the converted external acceleration vector and a correction factor.

17. The method of claim 16, further comprising generating a position of the lower limb prosthetic device based on the velocity of the lower limb prosthetic.

18. (canceled)

19. The method of claim 16, further comprising generating the correction factor based on a gait state of the lower limb prosthetic device.

20. The method of claim 19, wherein the gait state comprises a stationary state, a single supported state or an others state.

21. The method of claim 20, wherein if the gait state is the stationary state or the single supported state, the correction factor is determined based on a correction factor of a previous gait cycle, wherein if the gait state is the others state, the correction factor is a correction factor determined in a previous single supported state.

22. The method of claim 19, further comprising updating the correction factor when the lower limb prosthetic is in a single supported stance phase.

23. (canceled)

24. (canceled)

25. (canceled)

26. (canceled)
